(19) United States
(12) Patent Application Publication
(10) Pub. No.: US 2007/0213686 A1
(43) Pub. Date: Sep. 13, 2007

(54) System and method for prevention of radiocontrast induced nephropathy

(75) Inventors: Vandana S. Mathur, Woodside, CA (US); Craig A. Ball, San Carlos, CA (US); Jeffrey M. Elkins, Novato, CA (US); Harry B. Goodson IV, Fremont, CA (US); Samir R. Patel, Mountain View, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: FlowMedica, Inc., Fremont, CA (US)

(21) Appl. No.: 11/347,008

(22) Filed: Feb. 3, 2006

Related U.S. Application Data
(63) Continuation of application No. PCT/US03/29586, filed on Sep. 22, 2003.
(60) Provisional application No. 60/493,100, filed on Aug. 5, 2003.
(60) Provisional application No. 60/502,468, filed on Sep. 13, 2003.

(51) Int. Cl. A61B 19/00

(52) U.S. Cl. 604/518; 128/898; 604/151; 604/500; 604/93.01

(57) ABSTRACT

An apparatus and method particularly useful in treatments and therapies directed at the kidneys such as the prevention of radiocontrast nephropathy (RCN) arising from diagnostic procedures using iodinated contrast materials. A series of treatment schemes are provided based upon local therapeutic agent delivery to the kidneys that can be used as a prophylactic treatment for patients undergoing interventional procedures that have been identified as being at an elevated risk for developing RCN as well as for low risk patients. The methods may include pre-exposure and post contrast exposure treatments alone or in combination with the local delivery of therapeutic agents to the kidneys. Among the agents identified for such treatments are normal saline and the vasodilators papaverine and fenoldopam mesylate and appropriate dosing is provided.

### CROSS-REFERENCE TO RELATED APPLICATIONS

This application claims priority from U.S. provisional application Ser. No. 60/493,100 filed on Aug. 5, 2003, incorporated herein by reference in its entirety.

This application claims priority from U.S. provisional application Ser. No. 60/502,468 filed on Sep. 13, 2003 via Express Mail No. EV352305142US, incorporated herein by reference in its entirety.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### BACKGROUND OF THE INVENTION

1. Field of the Invention

This invention pertains generally to systems and methods for providing local therapy to renal systems in patients, and more particularly to a system and method for treating renal conditions with local delivery of fluid agents to the renal system.

2. Description of Related Art

Each year large numbers of patients are exposed to contrast media associated with diagnostic imaging and treatment procedures. Various diagnostic systems and procedures have been developed using local delivery of dye (e.g. radiopaque “contrast” agent) or other diagnostic agents, that allow an external monitoring system to gather important physiological information based upon the diagnostic agent's movement or assimilation in the body at the location of delivery and/or at other locations affected by the delivery site. Angiography is one such practice using a hollow, tubular angiography catheter for locally injecting radiopaque dye into a blood chamber or vessel, such as for example coronary arteries in the case of coronary angiography, or in a ventricle in the case of cardiac ventriculography.

Other systems and methods have been disclosed for locally delivering a therapeutic agent into a particular body tissue within a patient via a body lumen. For example, angiographic catheters of the type just described above, and other similar tubular delivery catheters, have also been disclosed for use in locally injecting treatment agents through their delivery lumens into such body spaces within the body. More detailed examples of this type include local delivery of thrombolytic drugs such as TPA™, heparin, cumadin, or urokinase into areas of existing clot or thrombogenic implants or vascular injury. In addition, various balloon catheter systems have also been disclosed for local administration of therapeutic agents into target body lumens or spaces, and in particular associated with blood vessels. More specific previously disclosed of this type include balloons with porous or perforated walls that elute drug agents through the balloon wall and into surrounding tissue such as blood vessel walls.

Yet further examples for localized delivery of therapeutic agents include various multiple balloon catheters that have spaced balloons that are inflated to engage a lumen or vessel wall in order to isolate the intermediate catheter region from in-flow or out-flow across the balloons. According to these examples, a fluid agent delivery system is often coupled to this intermediate region in order to fill the region with agent such as drug that provides an intended effect at the isolated region between the balloons.

The diagnosis or treatment of many different types of medical conditions associated with various different systems, organs, and tissues, may also benefit from the ability to locally deliver fluids or agents in a controlled manner. In particular, various conditions related to the renal system would benefit a great deal from an ability to locally deliver of therapeutic, prophylactic, or diagnostic agents into the renal arteries.

Acute renal failure (“ARF”) is an abrupt decrease in the kidney's ability to excrete waste from a patient's blood. This change in kidney function may be attributable to many causes. A traumatic event, such as hemorrhage, gastrointestinal fluid loss, or renal fluid loss without proper fluid replacement may cause the patient to go into ARF. Patients may also become vulnerable to ARF after receiving anesthesia, surgery, or α-adrenergic agonists because of related systemic or renal vasoconstriction. Additionally, systemic vasodilation caused by anaphylaxis, and anti-hypertensive drugs, sepsis or drug overdose may also cause ARF because the body's natural defense is to shut down, i.e., vasoconstriction of non-essential organs such as the kidneys. Reduced cardiac output caused by cardiogenic shock, congestive heart failure, pericardial tamponade or massive pulmonary embolism creates an excess of fluid in the body, which can exacerbate congestive heart failure. For example, a reduction in blood flow and blood pressure in the kidneys due to reduced cardiac output can in turn result in the retention of excess fluid in the patient's body, leading, for example, to pulmonary and systemic edema.

The renal system in many patients may also suffer from a particular fragility, or otherwise general exposure, to potentially harmful effects of other medical device interventions. For example, the kidneys as one of the body's main blood filtering tools may suffer damage from exposed to high-density radiopaque contrast dye, such as during coronary, cardiac, or neuro-angiography procedures. One particularly harmful condition known as “radiocontrast nephropathy” or “RCN” is often observed during such procedures, wherein an acute impairment of renal function follows exposure to such radiographic contrast materials, typically resulting in a rise in serum creatinine levels of more than 25% above baseline, or an absolute rise of 0.5 mg/dl within 48 hours. Therefore, in addition to congestive heart failure (CHF), renal damage associated with RCN is also a frequently observed cause of ARF. Radiocontrast induced nephropathy is one of the most common causes of hospital onset renal failure and renal impairment in hospital patients. While most patients recover the majority of renal function, a minority become dialysis dependant,

In addition, the proper function of the kidney is directly related to cardiac output and related blood pressure into the renal system. These physiological parameters, as in the case of congestive heart failure (CHF), may also be significantly compromised during a surgical intervention such as an angioplasty, coronary artery bypass, valve repair or replacement, or other cardiac interventional procedure. A patient undergoing these procedures may be particularly susceptible to renal damage from contrast imaging.

There would be great advantage therefore from an ability to locally deliver drugs and other prophylactic materials directly into the renal arteries contemporaneously with, with surgical interventions, as well as with radiocontrast dye delivery. However, many such procedures are conducted with medical device systems, such as using guiding catheters or angiography catheters having outer dimensions typically ranging between about 4 French to about 12 French, and ranging generally between about 6 French to about 8 French in the case of guide catheter systems for delivering angioplasty or stent devices into the coronary or neurovascular arteries (e.g. carotid arteries). These devices also are most typically delivered to their respective locations for use (e.g. coronary ostia) via a percutaneous, translumenal access in the femoral arteries and retrograde delivery upstream along the aorta past the region of the renal artery ostia. A Seldinger access technique to the femoral artery involves relatively controlled dilation of a puncture hole to minimize the size of the intruding window through the artery wall, and is a preferred method where the profiles of such delivery systems are sufficiently small. Otherwise, for larger systems a “cut-down” technique is used involving a larger, surgically made access window through the artery wall.

Accordingly, a local renal agent delivery system for contemporaneous use with other retrogradedly delivered medical device systems, such as of the types just described above, would preferably be adapted to allow for such interventional device systems, in particular of the types and dimensions just described, to pass upstream across the renal artery ostia (a) while the agent is being locally delivered into the renal arteries, and (b) while allowing blood to flow downstream across the renal artery ostia, and (c) in an overall cooperating system that allows for Seldinger femoral artery access. Each one of these features (a), (b), or (c), or any sub-combination thereof, would provide significant value to patient treatment; a local renal delivery system providing for the combination of all three features is particularly valuable.

Notwithstanding the clear needs for and benefits that would be gained from such local drug delivery into the renal system, the ability to do so presents unique challenges. In one regard, the renal arteries extend from respective ostia along the abdominal aorta that are significantly spaced apart from each other circumferentially around the relatively very large aorta. Often, these renal artery ostia are also spaced from each other longitudinally along the aorta with relative superior and inferior locations. This presents a unique challenge to locally deliver drugs or other agents into the renal system on the whole, which requires both kidneys to be fed through these separate respective arteries via their uniquely positioned and substantially spaced apart ostia. This becomes particularly important where both kidneys may be equally at risk, or are equally compromised, during an invasive upstream procedure—or, of course, for any other indication where both kidneys require local drug delivery. Thus, an appropriate local renal delivery system for such indications would preferably be adapted to feed multiple renal arteries perfusing both kidneys.

In another regard, mere local delivery of an agent into the natural, physiologic blood flow path of the aorta upstream of the kidneys may provide some beneficial, localized renal delivery versus other systemic delivery methods, but various undesirable results still arise. In particular, the high flow aorta immediately washes much of the delivered agent beyond the intended renal artery ostia. This reduces the amount of agent actually perfusing the renal arteries with reduced efficacy, and thus also produces unwanted loss of the agent into other organs and tissues in the systemic circulation (with highest concentrations directly flowing into downstream circulation).

In still a further regard, various known types of tubular local delivery catheters, such as angiographic catheters, other “end-hole” catheters, or otherwise, may be positioned with their distal agent perfusion ports located within the renal arteries themselves for delivering agents there, such as via a percutaneous translumenal procedure via the femoral arteries (or from other access points such as brachial arteries, etc.). However, such a technique may also provide less than completely desirable results.

For example, such seating of the distal tip of the delivery catheter within a renal artery may be difficult to achieve from within the large diameter/high flow aorta, and may produce harmful intimal injury within the artery. Also, where multiple kidneys must be infused with agent, multiple renal arteries must be cannulated, either sequentially with a single delivery device, or simultaneously with multiple devices. This can become unnecessarily complicated and time consuming and further compound the risk of unwanted injury from the required catheter manipulation. Moreover, multiple dye injections may be required in order to locate the renal ostia for such catheter positioning, increasing the risks associated with contrast agents on kidney function (e.g. RCN)—the very organ system to be protected by the agent delivery system in the first place. Still further, the renal arteries themselves, possibly including their ostia, may have pre-existing conditions that either prevents the ability to provide the required catheter seating, or that increase the risks associated with such, mechanical intrusion. For example, the artery wall may be diseased or stenotic, such as due to atherosclerotic plaque, clot, dissection, or other injury or condition. Finally, among other additional considerations, previous disclosures have yet to describe an efficacious and safe system and method for positioning these types of local agent delivery devices at the renal arteries through a common introducer or guide sheath shared with additional medical devices used for upstream interventions, such as angiography or guide catheters. In particular, to do so concurrently with multiple delivery catheters for simultaneous infusion of multiple renal arteries would further require a guide sheath of such significant dimensions that the preferred Seldinger vascular access technique would likely not be available, instead requiring the less desirable “cut-down” technique.

In addition to the various needs for locally delivering agents into branch arteries described above, much benefit may also be gained from simply locally enhancing blood perfusion into such branches, such as by increasing the blood pressure at their ostia. In particular, such enhancement would improve a number of medical conditions related to insufficient physiological perfusion into branch vessels, and in particular from an aorta and into its branch vessels such as the renal arteries.

Certain prior disclosures have provided surgical device assemblies and methods intended to enhance blood delivery into branch arteries extending from an aorta. For example, intra-aortic balloon pumps (IABPs) have been disclosed for use in diverting blood flow into certain branch arteries. One such technique involves placing an IABP in the abdominal aorta so that the balloon is situated slightly below (proximal to) the branch arteries. The balloon is selectively inflated and deflated in a counter pulsation mode (by reference to the physiologic pressure cycle) so that increased pressure distal to the balloon directs a greater portion of blood flow into principally the branch arteries in the region of their ostia. However, the flow to lower extremities downstream from such balloon system can be severely occluded during portions of this counter pulsing cycle. Moreover, such previously disclosed systems generally lack the ability to deliver a drug or agent to the branch arteries while allowing continuous and substantial downstream perfusion sufficient to prevent unwanted ischemia.

It is further noted that, despite the renal risks described in relation to radiocontrast dye delivery, and in particular RCN, in certain circumstances local delivery of such dye or other diagnostic agents is indicated specifically for diagnosing the renal arteries themselves. For example, diagnosis and treatment of renal stenosis, such as due to atherosclerosis or dissection, may require dye injection into a subject renal artery. In such circumstances, enhancing the localization of the dye into the renal arteries may also be desirable. In one regard, without such localization larger volumes of dye may be required, and the dye lost into the downstream aortic flow may still be additive to impacting the kidney(s) as it circulates back there through the system. In another regard, an ability to locally deliver such dye into the renal artery from within the artery itself, such as by seating an angiography catheter there, may also be hindered by the same stenotic condition requiring the dye injection in the first place (as introduced above). Still further, patients may have stent-grafts that may prevent delivery catheter seating.

Notwithstanding the interest and advances toward locally delivering agents for treatment or diagnosis of organs or tissues, the previously disclosed systems and methods summarized immediately above generally lack the ability to effectively deliver agents from within a main artery and locally into substantially only branch arteries extending therefrom while allowing the passage of substantial blood flow and/or other medical devices through the main artery past the branches. This is in particular the case with previously disclosed renal treatment and diagnostic devices and methods, which do not adequately provide for local delivery of agents into the renal system from a location within the aorta while allowing substantial blood flow continuously downstream past the renal ostia and/or while allowing distal medical device assemblies to be passed retrogradedly across the renal ostia for upstream use. Much benefit would be gained if agents, such as protective or therapeutic drugs or radiopaque contrast dye, could be delivered to one or both of the renal arteries in such a manner.

Several more recently disclosed advances have included local flow assemblies using tubular members of varied diameters that divide flow within an aorta adjacent to renal artery ostia into outer and inner flow paths substantially perfusing the renal artery ostia and downstream circulation, respectively. Such disclosures further include delivering fluid agent primarily into the outer flow path for substantially localized delivery into the renal artery ostia. These disclosed systems and methods represent exciting new developments toward localized diagnosis and treatment of pre-existing conditions associated with branch vessels from main vessels in general, and with respect to renal arteries extending from abdominal aortas in particular.

However, while these approaches in one regard provide benefit by removing the need to cannulate each renal artery of the bi-lateral renal system, substantial benefit would still be gained conversely from a device system and method that allows for direct bilateral renal artery infusion without the need to deploy flow diverters or isolators into the high-flow abdominal aorta. In one particular example, patients that suffer from abdominal aortic aneurysms may not be suitable for standard delivery systems with flow diverters or isolators that are sized for normal arteries. In another regard, direct renal artery infusion allows for reduced occlusion to downstream aortic blood flow, or conversely more downstream flow may be preserved. Still further, the ability to truly isolate drug to only the renal system, without the potential for downstream leaking or loss into the systemic circulation, may be maximized.

A need therefore still exists for improved systems and methods for locally delivering agents bilaterally into each of two renal arteries perfusing both kidneys of a patient while a substantial portion of aortic blood flow is allowed to perfuse downstream across the location of the renal artery ostia and into the patient's lower extremities.

A need still exists for improved systems and methods for efficiently gaining percutaneous translumenal access into each side of the kidney system via their separate renal artery ostia along the abdominal aortic wall, so that procedures such as fluid agent delivery may be performed locally within both sides of the renal system.

A need still exists for improved systems and methods for locally delivering fluid agents into a renal artery from a location within the aorta of a patient adjacent the renal artery's ostium along the aorta wall.

A need still exists for improved systems and methods for locally isolating delivery of fluids or agents into the renal arteries of a patient, and while allowing other treatment or diagnostic devices and systems, such as angiographic or guiding catheter devices and related systems, to be delivered across the location.

A need still exists for improved systems and methods for locally delivering fluids or agents into the renal arteries of a patient, for prophylaxis or diagnostic procedures related to the kidneys.

A need still exists for improved systems and methods for locally isolating delivery of fluids or agents into the renal arteries of a patient in order to treat, protect, or diagnose the renal system adjunctive to performing other contemporaneous medical procedures such as angiograms other translumenal procedures upstream of the renal artery ostia.

A need still exists for improved systems and methods for delivering both a local renal drug delivery system and at least one adjunctive distal interventional device, such as an angiographic or guiding catheter, through a common delivery sheath.

A need also still exists for improved systems and methods for delivering both a local renal drug delivery system and at least one adjunctive distal interventional device, such as an angiographic or guiding catheter, through a single access site, such as a single femoral arterial puncture.

A need also still exists for improved systems and methods for treating, and in particular preventing, ARF, and in particular relation to RCN or CHF, by locally delivering renal protective or ameliorative drugs into the renal arteries, such as contemporaneous with radiocontrast injections such as during angiography procedures.

### BRIEF SUMMARY OF THE INVENTION

Radiocontrast induced nephropathy is a common cause of treatment related renal failure or diminished function. It is believed that the condition that is likely to be responsible for damaging renal function is contrast induced renal tubular ischemia. In addition, direct toxicity from contact with contrast media and the appearance of free radicals may contribute to the development of radiocontrast nephropathy.

According to one aspect of the present invention, systems and methods are provided for prophylactic and post exposure treatments to the kidneys and renal vasculature to maintain kidney function after radiocontrast exposure.

According to another aspect of the invention, a local bilateral renal therapy delivery system and a therapeutic dose of a renal therapy agent is provided that is adapted to deliver the therapeutic dose to each of two renal arteries having unique respective renal ostia along an abdominal aorta wall in the patient.

Another aspect of the invention provides a method for preventing radiocontrast induced nephropathy in a patient by delivering a first therapeutic dose of a first renal therapy agent to the patient during a first period that is before exposure to a radiocontrast agent; and then locally delivering a second therapeutic dose of a second renal therapy agent bi-laterally to the renal arteries of the patient during a second period that is during exposure to the radiocontrast agent.

Still a further aspect of the invention provides a method that locally delivers a first therapeutic dose of a first renal therapy agent bi-laterally to the renal arteries of the patient during exposure to radio contrast and then systemically delivering a second therapeutic dose of a second renal therapy agent as a tail after exposure to the radiocontrast.

According to another aspect of the invention, a method is provided that locally delivers a first therapeutic dose of a first therapeutic agent bi-laterally to the renal arteries of a patient during exposure to a radiocontrast agent and then locally delivers a second therapeutic dose of a second therapeutic agent bi-laterally to the renal arteries of the patient subsequent to exposure to the radiocontrast agent.

Another aspect of the invention provides method for preventing radiocontrast-induced nephropathy by systemically delivering a first therapeutic dose of a first renal therapy agent to the patient during before delivery of the radiocontrast agent to the patient. A second therapeutic dose of a renal therapy agent is then locally delivered to the patient during exposure to the radiocontrast agent and then a third therapeutic dose of a renal therapy agent to the patient is systemically delivered to the patient after exposure to the radiocontrast agent.

Yet another aspect of the invention provides a system for protecting a renal system from radiocontrast nephropathy associated with delivery of a radiocontrast agent within a vascular system of a patient that has a kit with a bi-lateral local renal therapy system; a source of fluid agent; a pre-printed instructions for use (IFU). The bilateral local renal therapy system is adapted to couple to the source of fluid agent externally of the patient and to deliver a volume of fluid agent from the source and simultaneously into each of two renal arteries perfusing each of two kidneys, respectively, in the patient. The IFU comprises instructions providing a first therapeutic dose regimen related to local bilateral renal delivery of the volume of fluid agent with the bilateral local renal therapy system during a procedural phase wherein the radiocontrast agent is being delivered to a patient, and also providing a second therapeutic dose regimen related to bilateral renal delivery of the fluid agent during a second phase that is either a pre- or post-procedural phase that is temporally before or after, respectively, the procedural phase of radiocontrast agent delivery to the patient. The operation of the system according to the IFU is adapted to substantially protect the renal system from RCN associated with the radiocontrast delivery to the patient.

The various embodiments herein described for the present invention can be particularly useful in treatments and therapies directed at the kidneys such as the prevention of radiocontrast nephropathy (RCN) from diagnostic treatments using iodinated contrast materials. As a prophylactic treatment method for patients undergoing interventional procedures that have been identified as being at elevated risk for developing RCN, a series of treatment schemes have been developed based upon local therapeutic agent delivery to the kidneys. The treatment schemes may also be used with low risk patients. Among the agents identified for such treatment are normal saline (NS) and the vasodilators papaverine (PAP) and fenoldopam mesylate (FM).

The approved use for fenoldopam is for the in-hospital intravenous treatment of hypertension when rapid, but quickly reversible, blood pressure lowering is needed. Fenoldopam causes dose-dependent renal vasodilation at systemic doses as low as approximately 0.01 mcg/kg/min through approximately 0.5 mcg/kg/min IV and it increases blood flow both to the renal cortex and to the renal medulla. Due to this physiology, fenoldopam may be utilized for protection of the kidneys from ischemic insults such as high-risk surgical procedures and contrast nephropathy. Dosing from approximately 0.01 to approximately 3.2 mcg/kg/min is considered suitable for most applications of the present embodiments, or about 0.005 to about 1.6 mcg/kg/min per renal artery (or per kidney). As before, it is likely beneficial in many instances to pick a starting dose and titrate up or down as required to determine a patient's maximum tolerated systemic dose. Recent data, however, suggest that about 0.2 mcg/kg/min of fenoldopam has greater efficacy than about 0.1 mcg/kg/min in preventing contrast nephropathy and this dose is preferred.

The dose level of normal saline delivered bilaterally to the renal arteries may be set empirically, or beneficially customized such that it is determined by titration. The catheter or infusion pump design may provide practical limitations to the amount of fluid that can be delivered; however, it would be desired to give as much as possible, and is contemplated that levels up to about 2 liters per hour (about 25 cc/kg/hr in an average about 180 lb patient) or about one liter or 12.5 cc/kg per hour per kidney may be beneficial.

Local dosing of papaverine of up to about 4 mg/min through the bilateral catheter, or up to about 2 mg/min has been demonstrated safety in animal studies, and local renal doses to the catheter of about 2 mg/min and about 3 mg/min have been shown to increase renal blood flow rates in human subjects, or about 1 mg/min to about 1.5 mg/min per artery or kidney. It is thus believed that local bilateral renal delivery of papaverine will help to reduce the risk of RCN in patients with pre-existing risk factors such as high baseline serum creatinine, diabetes mellitus, or other demonstration of compromised kidney function.

It is also contemplated according to further embodiments that a very low, systemic dose of papaverine may be given, either alone or in conjunction with other medical management such as for example saline loading, prior to the anticipated contrast insult. Such a dose may be on the order for example of between about 3 to about 14 mg/hr (based on bolus indications of approximately 10-40 mg about every 3 hours—papaverine is not generally dosed by weight). In an alternative embodiment, a dosing of about 2-3 mg/min or about 120-180 mg/hr. Again, in the context of local bilateral delivery, these are considered halved regarding the dose rates for each artery itself.

Notwithstanding the particular benefit of this dosing range for each of the aforementioned compounds, it is also believed that higher doses delivered locally would be safe. Titration is a further mechanism believed to provide the ability to test for tolerance to higher doses. In addition, it is contemplated that the described therapeutic doses can be delivered alone or in conjunction with systemic treatments such as intravenous saline.

Further aspects of the invention will be brought out in the following portions of the specification, wherein the detailed description is for the purpose of fully disclosing preferred embodiments of the invention without placing limitations thereon.

### BRIEF DESCRIPTION OF THE SEVERAL VIEWS OF THE DRAWING(S)

The invention will be more fully understood by reference to the following drawing which is for illustrative purposes only:

FIG. 1 is a flow diagram of the method of one embodiment according to the present invention.

FIG. 2 is a block diagram of pre-intervention, intervention and post intervention dosing schemes of normal saline, Fenoldopam and Papaverine according to the present invention.

FIG. 3 is a schematic drawing of an intra-renal artery delivery catheter particularly suited for delivery of the dosing schemes according to the present invention.

### DETAILED DESCRIPTION OF THE INVENTION

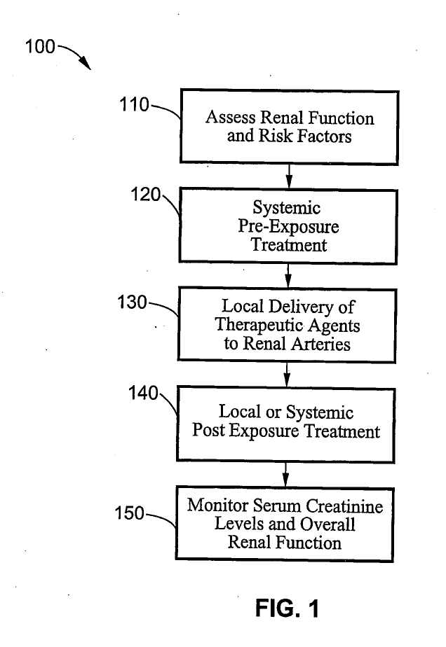

Referring more specifically to the drawings, for illustrative purposes the present invention is embodied in the apparatus and method generally shown in FIG. 1 through FIG. 3. It will be appreciated that the apparatus may vary as to configuration and as to details of the parts, and that the method may vary as to the specific steps and sequence, without departing from the basic concepts as disclosed herein.

It will also be appreciated that the various embodiments of the present invention relate to local fluid delivery to the renal system, and therefore relate to the use of local delivery devices to achieve such localized delivery. In general, such delivery may be accomplished unilaterally, e.g. into one “side” of the renal system, which generally treats conditions associated with one kidney or its vasculature or related tissues. Or, the local therapy may be accomplished bi-laterally, e.g. into both “sides” to treat both kidneys or related vasculature or related tissues. Providing localized therapy to a “side” of the renal system generally involves delivery via a renal artery on that side, typically through its renal ostium along the abdominal aorta wall. Typically each side is perfused by the abdominal aortic blood flow through each of two such ostia, respectively, that are spaced about the circumference of the abdominal aorta wall.

Therefore, devices may include for example intra-aortic delivery devices that inject fluids into the abdominal aorta in a manner such that those fluids flow principally into the one or both renal arteries via the corresponding ostium or ostia, depending upon whether single-sided or “bi-lateral” delivery is to be achieved. Or, therapy may be provided by a more direct approach, wherein the renal artery itself is cannulated with a delivery device, such as in percutaneous translumenal procedures via the renal ostium in a delivery approach through the abdominal aorta.

Particular challenges exist for providing local bi-lateral renal therapy with fluid delivery, and thus only recent developments have been directed toward accomplishing this goal. However, where the renal condition to be treated relates to the renal “system”, simultaneous therapy to both sides is often desirable, and thus device systems designed for such therapy are of substantial benefit.

Accordingly, while many different devices may be used to accomplish the various goals and methods of the particular embodiments herein described, it is in particular contemplated that such may be achieved in a beneficial matter according to various of the local renal therapy device systems and methods described in one or more of the following published PCT International Patent Applications: PCT/US00/00636 filed Nov. 1, 2000; PCT/US01/13686 filed Apr. 27, 2001, publication WO 01/083016 A3 published Nov. 8, 2001, and PCT/US03/21406 filed Jul. 9, 2003. Further examples of device systems and methods suitable for use in combination with the present embodiments are disclosed in the following pending U.S. Patent Application(s): U.S. Ser. No. 10/251,915 filed Sep. 20, 2002. Furthermore, other examples for suitable combination herewith are also disclosed in the following pending U.S. provisional patent applications: 09/229,390 filed on Jan. 11, 1999; 09/562,493 filed May 1, 2000; 09/724,691 filed Nov. 28, 2000; 60/412,343 filed Sep. 20, 2002; 60/412,476 filed Sep. 20, 2002; 60/476,347 filed Jun. 5, 2003; 60/479,329 filed Jun. 17, 2003, 06/486,349 and 60/486,206 Jul. 10, 2003. The disclosures of all these references noted above are hereby incorporated by reference herein in their entirety.

Whereas various medical conditions and indications may be suitable environments for using the various embodiments herein disclosed, the present invention is in particular beneficially applied for delivering therapeutic agents to the renal arteries of a patient who is simultaneously undergoing a coronary intervention or other therapy where radiocontrast dye injections are made. More specifically, the therapeutic agents delivered according to the embodiments thus function to protect the kidneys or increase the ability of the kidneys to process organically-bound iodine (radiographic contrast), as measured by serum creatinine and glomerular filtration rate (GFR).

It is to be appreciated that the terms “agent,” “drug,” “fluid” and “therapeutic dose” are frequently used throughout this disclosure for the purpose of explaining various aspects of the several embodiments. In general, the term “fluid” is intended to be given its ordinary meaning, and is generally described as a material that flows. The term “agent” may represent many different types of materials, including fluids, but may be otherwise such as powders, gels, suspensions, etc. In general, however, “agent” is intended to mean a material that when delivered has a generally useful effect in providing therapy. The term “drug” is generally used to describe regulated materials having the particular bioactivity of consequence to host organisms or their tissues. The term “drug” or “agent” is also intended to encompass compounds that, under physiological conditions, are converted into the therapeutically active agents. For example, selected moieties of an agent may be hydrolyzed under physiological conditions to provide the desired molecule. Alternatively, the agent or drug may be converted by the enzymatic activity of the body. The term “therapeutic dose” as used herein means that amount of a compound; material, drug or composition that is effective for producing some desired therapeutic effect either systemically or to specific organs or tissues of the body. In general, a suitable dose will be that amount of the compound that is the lowest dose effective to produce a therapeutic effect. Such an effective dose will generally depend upon the factors described above. In addition, the effective dose of the active compound may be administered as two, three, four, five, six or more sub-doses administered separately at appropriate intervals throughout the procedure preferably in unit dosage forms. In addition, a dose may mean a total quantity of a therapeutic agent administered over the course of a treatment or may be described in terms of a rate of introduction to a local area such as the renal arteries. The term “treatment” is intended to encompass prophylaxis, as well as local or systemic therapy or cure.

It is to be appreciated that, notwithstanding these helpful distinctions, these terms are generally intended to be interchangeable with respect to the context of their use in describing such specified embodiments or aspects of the invention hereunder. Where embodiments are described in the context of one of these terms is used, it is contemplated that such embodiments may also be described in the context of one or both of the others of these terms, unless otherwise specified to be exclusive of the others.

A significant portion of the patient population undergoing interventional procedures is at risk for developing contrast-induced nephropathy (RCN), the consequences of which include significant morbidity and even mortality. There is a need for selective local renal drug delivery because many of the drugs thought to be effective are known to have, or may have, harmful systemic effects (such as lowering of systemic blood pressure) if delivered systemically in sufficiently large doses to achieve the desired local effects. One particular example of this is vasodilators, wherein vasodilation may be a very desired effect to treat particular renal conditions, but system wide vasodilation may present serious complications.

Therefore, there exists a real clinical need in the interventional setting for a means to mitigate the risk of the onset of RCN. Patients requiring such mitigation can usually be identified by risk factors as indicated below, and thus prophylactic treatment may be limited to those patients in which there is a clearly established need in terms of propensity to develop RCN. Presented herein is a method for performing such prophylactic measures, including specific therapeutic preparations and methods, and associated devices for use with said methods.

As a prophylactic treatment method for patients undergoing interventional procedures that have been identified as being at elevated risk for developing RCN, a series of treatment schemes have been developed based upon local agent delivery to the kidneys. Among the agents identified for such treatment are normal saline (NS) and the vasodilators papaverine (PAP) and fenoldopam mesylate (FM).

Turning now to FIG. 1, one embodiment of the method 100 for preventing radiographic contrast induced reductions in renal function is shown. At block 110, the renal function and risk factors of a patient to radiographic contrast exposure are assessed.

It has been seen that the adverse effects of contrast usage are more severe in patients with pre-existing renal insufficiency, as in, for example, cases of congestive heart failure (CHF) or diabetes mellitus. In addition, patients with advanced age, dehydration, hyperuricemia, and prior renal problems are also at higher risk. Diabetics that are being treated with Meffermin may run a particular risk for lactacidosis and renal failure from contrast exposure while receiving such treatments. Patients with increased baseline serum creatinine levels also have an increased risk for developing radiocontrast-induced neuropathy.

Radio opaque contrast agents containing organically bound iodine, and routinely used in interventional cardiology and radiology procedures, are known to cause in some cases acute and permanent functional impairment to the renal system. The primary radiocontrast agents used for medicinal diagnostics are 2,4,6-Triiodobenzoic acid derivatives. Iodinated contrast media are classified into ionic and non-ionic categories depending on the chemical structure and ratios of ions to iodine atoms. Non-ionic contrast agents have been shown to have a lower incidence of adverse reactions generally but are substantially more expensive than ionic contrast agents. Consequently, non-ionic agents are typically reserved for use with higher risk patients.

Radio contrast-induced nephropathy is characterized by a rise in serum creatinine (SC) levels of at least 25%. Although serum creatinine is itself a surrogate marker of overall renal health, it has a proven history as a reliable overall assessment of the ability of the kidneys of the patient to process waste in an efficient manner, being very well correlated in the medical literature and in current practice with overall renal health.

Radiocontrast agents reduce renal function by altering the hemodynamics of the kidneys as well as exhibiting direct toxic effects on the tubular epithelial cells. There is also evidence that creation of reactive oxygen species (free radicals) may contribute to the damage caused by contrast agents.

Specifically, the contrast agent causes the greatest insult to the inner medullary region of the kidney. This region of the kidney normally functions on the edge of hypoxia, due to both the high metabolic needs of filtration (namely active transport of sodium ions) and low P_{O2}, a byproduct of the countercurrent exchange system that allows the concentration of urine. Iodinated contrast media cause a generally minor constrictive reaction to the vascular system generally, which manifests itself in undesirable clinical sequelae with respect to the medullary kidney for the hypoxic reasons mentioned above. Filtration of the contrast media out of the bloodstream is relatively quick (on the order of minutes), owing to the low osmolarity of most contrast agents in use clinically today and the large volume of blood filtered by the kidneys in each cardiovascular cycle. However, the resultant constrictive effect continues for some period (hours) after the contrast has been removed.

Accordingly, it will be seen that the risk assessment may determine the type of contrast agent that is used as well as the pre, during and post procedure treatments that may be given to a patient.

Referring now to block 120 of FIG. 1, a systemic prophylactic treatment is optionally conducted prior to the introduction of contrast and the primary procedure is shown. It is highly beneficial that this treatment result in an increase to the effective circulating volume of blood in the system. For example, the systemic administration of normal saline is one beneficial mode of this aspect.

Saline is considered to be the standard of care for prevention of contrast nephropathy and has been shown to be superior to numerous other systemic strategies for the prevention of contrast nephropathy including orally or systemically administered agents known in the art. Saline is also non-toxic and an effective prophylaxis against other agents that induce acute renal failure by causing renal vasoconstriction (i.e. amphotericin B).

Although the normal saline is typically administered intravenously, there is considerable collective experience with medical practitioners with intra-arterial administration of saline. For example, with arterial flushes during invasive vascular procedures, it is not unusual to administer normal saline at the rate of 300 mL/hr. Further, normal saline may also be administered into the arterial side of a hemodialysis circuit for purposes of volume replacement.

However, because patients undergoing cardiac catheterization often have poor cardiac function, their ability to tolerate systemic saline loads may be limited. Further, because patients are often admitted and discharged on the same day for cardiac catheterization procedures, there is insufficient time to adequately saline load patients. Both of the above factors often results in an inadequate utilization of saline to help reduce the occurrence of contrast nephropathy.

As stated above, over-hydration of patients at elevated risk for developing radio contrast nephropathy may reduce their risk, whereas conversely arterial volume depletion increases the risk of contrast nephropathy. Saline delivery mitigates the risk of contrast nephropathy, in one regard, through increasing renal blood flow by causing volume expansion. Therefore, saline likely increases oxygen delivery to the kidneys. Saline may also help to “flush” out debris from damaged renal tubular cells and thereby prevent “back pressure” within the tubules that lead to reduced GFR in patients with acute renal failure. Finally, due to glomerulotubular feedback, higher delivery of sodium (Na) to the kidneys decreases the re-absorption of Na by the kidneys. Because the most energy demanding processes within the kidneys are those of tubular transport, saline may effectively reduce energy demands within the kidney.

Although the pre-procedure treatment with normal saline is considered highly beneficial, it will be understood that other systemic treatments may be administered. For example, physicians may concomitantly administer with saline one or more of the following: Dopamine, Mannitol, endothelin antagonists, atrial or B-type natriuretic peptide, N-acetylcysteine, calcium channel blockers, L-Arginine or theophylline and the like.

As shown at block 130 of FIG. 1, local delivery of therapeutic agents to the renal arteries is accomplished during the primary procedure in the embodiment shown. One or more delivery catheters are preferably positioned within or in the vicinity of the arterial system of the patient to deliver a discrete volume or a continuous volume of therapeutic agents to the renal arteries of each kidney. It is a consideration of the present invention that local delivery of agents to the kidneys, via vascular delivery into the renal arteries, will accentuate the effect of these agents (and by extension, possibly others) on the kidneys with substantially diminished (or no) adverse systemic repercussions.

It can be seen that the local delivery of smaller quantities of a therapeutic agent can create local concentrations to provide the desired physiological effect on the kidneys while avoiding the systemic side effects that occur from much larger doses delivered orally or intravenously. Likewise, greater concentrations of agents can be delivered to the kidneys than could normally be tolerated systemically.

For example, intra-arterial papaverine (PAP) delivery has been previously investigated for the intended immediate relief of vasospasm that occurs due to excess guide wire or catheter manipulation within a vessel. PAP is believed to have a positive effect on relieving the similarly artificially induced vasoconstriction caused by the contrast agents. In this manner, the medullary capillaries stay open despite the effects of contrast, or even open further, and thus allow for more blood flow through the tissue thereby preventing the hypoxic state from worsening and thus preserving the medullary tissue and its important biological function. However, continuous, systemic dosing of papaverine would clinically be quite dangerous, as severe systemic hypotension could quickly result if the patient was not monitored carefully. Local delivery of PAP, at higher locally therapeutic concentrations and lower systemic concentrations, avoids such systemic complications.

Similarly, with the example of fenoldopam mesylate (FM), local effects on the kidneys can be obtained with lower doses than can be tolerated systemically due to concerns of systemic hypotension. FM is a smooth muscle relaxant that acts directly on the capillary beds of the inner medulla. PAP, for example, is known to have a more general vasodilatory effect.

It will also be appreciated that treatment regimens using complimentary or synergistic therapeutic agents can be employed. The systemic treatment may complement or have a synergistic effect with the agent that is delivered locally.

For example, if a systemic infusion of normal saline is used in the clinical setting at block 120 of FIG. 1 to increase the systemic hydration level of a patient (which can be measured by central venous pressure, or CVP) in order to promote increased kidney function, complementary agents including additional normal saline can be administered to prevent the acute insult of radio contrast material to the medullary regions of the kidneys. It is known that the kidneys rapidly remove this excess fluid, and that fluid overload may be avoided by careful monitoring of a patient's CVP or via pulse oximetry. However, this does place a limit based on a given patient's baseline renal health of how much fluid can be tolerated, and the possibility exists that a clinically efficacious administration may be beyond a given patients' renal capacity.

Although normal saline, papaverine and fenoldopam mesylate are identified as beneficial for local delivery, other therapeutic agents can be used. For example, a list of various agents that may be considered bioactive with respect to renal function and with which the present embodiments may be suitably applied or suitably modified to provide appropriate renal therapy regimens, either for treatment of RCN or other renal conditions such as acute renal failure (ARF) concomitant with congestive heart failure (CHF), include: vasodilators, including for example papaverine, fenoldopam, calcium channel blockers, atrial natriuretic peptide (ANP), acetylcholine, nifedipine, nitroglycerine, nitroprusside, adenosine, dopamine, and theophylline; antioxidants, such as for example acetylcysteine; and agents, such as for example mannitol, or furosemide. Moreover, analogs or derivatives of these agents are contemplated, as are various combinations or blends thereof that may be considered beneficial to the renal therapy systems and methods herein described. Additionally, physicians may use non-ionic or iso-osmolar contrast with patients with a very high risk of contrast induced renal compromise in order to reduce the likelihood of renal injury.

After the primary treatment and introduction of radiocontrast to the blood stream, the contrast is freely filtered by the glomeruli and is neither secreted nor absorbed by the tubules and therefore has a half-life within the body. At block 140 of FIG. 1, a post procedure treatment is administered either locally or systemically or both. Vasodilators or other therapeutic agents introduced at block 130 of FIG. 1 may continue to be introduced after the primary procedure is completed until the contrast concentration levels are reduced.

Another example may be the introduction of antioxidants such as superoxide dismutase or acetylcysteine to quench any reactive oxygen species that may have been generated during the radiological imaging.

At block 150 of FIG. 1, the renal function and status of the patient is monitored to ensure the effectiveness of the treatments. In the embodiment shown, the renal function is monitored by determining serum creatinine levels. However, it will be understood that there are other diagnostic procedures in the art that can be an effective monitor of the renal function of the patient and can be used alternatively or in addition to serum creatinine levels. Ineffective or partially effective prophylactic measures and treatments may require further dosing, or other therapies such as the use of dialysis or other medical intervention to supplement or bolster renal function.

The invention according to the foregoing description and by reference to the accompanying figures therefore contemplates various embodiments of a broader therapeutic agent delivery regimen that utilizes bi-lateral local renal agent delivery, taking advantage of the abilities made possible by various new and beneficial delivery devices providing for such delivery in a manner that is manageable in conjunction with adjunctive procedures. In general, these aspects provide highly beneficial modalities by which local bi-lateral renal delivery may be completed for successful therapy. In addition, further modes are provided that provide overall procedural renal therapy in a manner that is customized over different periods of time surrounding a procedure. These different time periods are characterized by unique relative needs with respect to renal protection, as well as unique relative patient management environments, both aspects of patient care that play a role in certain of the embodiments described hereunder.

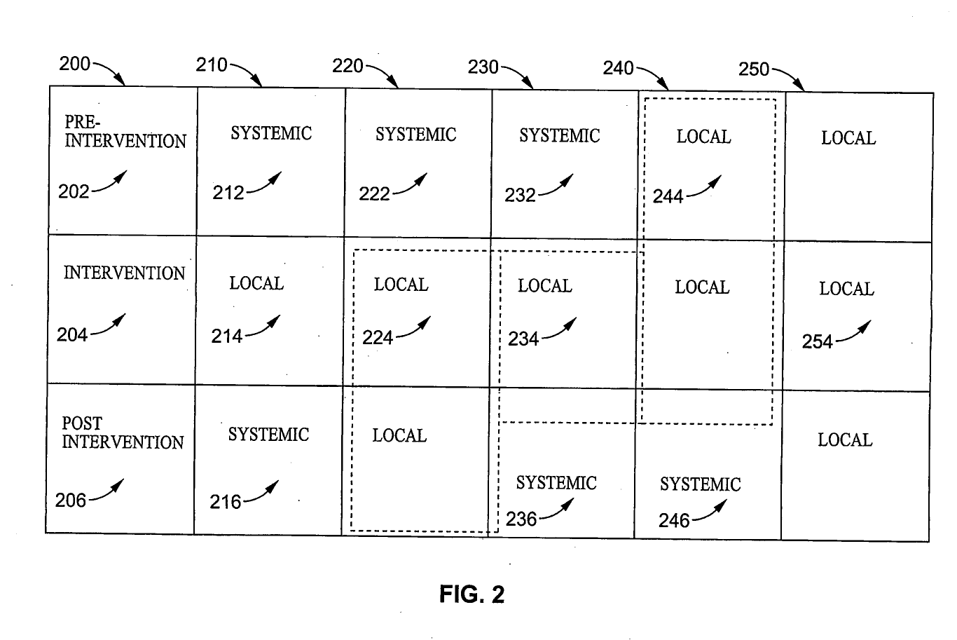

Various particular embodiments by which renal protection protocols may be customized at different periods in relation to an interventional procedure are further described by reference to FIG. 2. More specifically, the column designated at 200 shown schematically on the left side of FIG. 2 represents the following three windows of renal protective need with respect to interventional procedures: pre-intervention 202; intervention 204; and post-intervention 206. The various columns 210, 220, 230, 240, and 250 designate various different embodiments for managed renal protection as related to the interventional phases shown in column 200.

In one particular embodiment shown at column 210 in FIG. 2, pre-procedural renal therapy involves systemic agent delivery window 212 at the pre-intervention period 202, followed by a bi-lateral local renal delivery window 214 during the interventional procedure period 204, followed thereafter by a systemic delivery tail or window 216 during the post-procedural period 206. In the pre- and post-procedural periods 202,206, patients are not undergoing the high stress concentrations of dye loading to their kidneys that they experience during the interventional procedure period 204. Moreover, patients during this phase may not be in the operating room or catheter lab, or under constant caregiver supervision or monitoring. This protocol thus manages patients systemically during this time, such as using a simple IV drip. However, during the procedural period 204, when renal stress is highest immediately following dye injections, the renal protection management is more aggressive with bi-lateral local delivery doses per window 214. This is done with translumenal bi-lateral delivery catheters at a time when the patient is already on the catheter lab table, and already cannulated. In fact, according to certain highly beneficial device embodiments, common vascular access devices may be used with the local delivery system as well as the angiography system. According to the systemic-local-systemic tri-phasic therapy just described by reference to column 210 in FIG. 2, the solution is thus modified to meet the changing needs, and different patient management environments, at different times surrounding a procedure 200.

It is to be understood that despite the benefits just described, such tri-phasic protocol of the prior embodiment of column 210 may be modified to other combinations of therapeutic modalities within the different phases of the operation or procedure. In one particular regard, the post-procedural window remains a risk environment for the kidneys—the filtering process remains a stressful task despite the decaying load, and in particular to the extent the effects over time in some regards may become additive to the filters.

Therefore, a further embodiment shown in column 220 of FIG. 2 extends the local bi-lateral therapy window 214 beyond the period 204 during the invasive procedure, and into the post-procedural window 206. At this point, the patient's renal arteries would already be cannulated, so the task is only to maintain that cannulation longer for the purpose of extended agent infusion. While this may result in increased observation and associated healthcare cost, the benefit of longer local delivery at the higher local doses may be substantial in many cases.

In one particular further embodiment, this local delivery window 224 replaces the post-procedural systemic therapy window 216 of the prior embodiment, thus providing a biphasic therapy protocol that treats the post-procedural period 206 of similar import, and thus essentially as the same critical window, as interventional period 204 with respect to the need for renal protection.

In another particular further embodiment shown in column 230 of FIG. 2, while the local bilateral cannulation and renal delivery period 234 is extended beyond the interventional procedure 204, it nevertheless is thereafter replaced with the third systemic window 236 of renal protection, albeit later than the first instance of triphasic approach noted above with respect to the protocol of column 210.

In still a further regard illustrated by the embodiment of column 240 in FIG. 2, the local delivery window 244 is extended earlier in time than prior embodiments, as shown in the particular embodiment to replace the systemic phase of the pre-procedural period 202. This may require earlier cannulation, but due to the more concentrated local effects the renal therapy may be initiated a shorter period of time before the interventional procedure period 204. In this instance, systemic complications are minimized during the entire pre-interventional period and operation period. A systemic tail 246 is still shown in this embodiment during the post-procedural period, relieving the renal stress during this period 206 post-dye delivery, but again removing the need for on-going invasive cannulation such that the need for constant observation may be lightened. Nonetheless, it is still contemplated that in further embodiments an entire procedure may be done with local dose therapy, as shown in column 250 if FIG. 2.

Notwithstanding the various foregoing embodiments for mono-phasic, bi-phasic, or tri-phasic approaches to local renal delivery protocols and in conjoined relation with systemic therapies, the particular embodiments providing early systemic, peri-procedural local, and post-procedural systemic tail therapy are considered in particular beneficial for many cases of patient renal management.

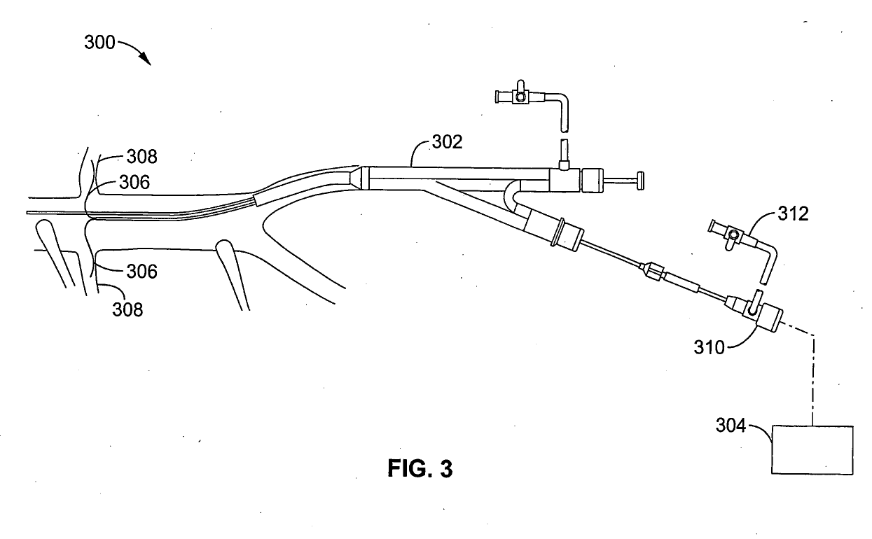

Referring now to FIG. 3, one embodiment of a bi-lateral intra-renal artery delivery system is generally shown for use with the dosing schemes disclosed in FIG. 2. The catheter system 300 may be provided in a kit form with a catheter 302 and a sufficient quantity of drug in a prepackaged container 304 for a single or multiple procedures. The pre-packaged container 304 for use according to the dosing methodology described herein can be sold or provided separately or in combination with the intra-renal delivery system provided in the kit. In addition, it will be understood that the pre-packaged drug or combination of drugs may be used with conventional catheters according to the dosing set forth herein.

In the embodiment shown in FIG. 3, the catheter 302 is inserted into the vessels of the body according to established protocols. The distal tips 306 of the catheter are disposed in the renal arteries 308. Therapeutic drugs are directed from vial 304 through a drug delivery port 310 through the catheter 302 and out of the distal tips 306 to the renal arteries 308. Distribution of the drug may be facilitated with the flow of saline through a saline port 312. It can be seen that the renal arteries 308 as well as the kidneys can be treated in this manner.

Various aspects of these embodiments just described may be better understood with reference to the accompanying more particular embodiments related to three specified types of renal therapeutic agents. These particular protocols are considered in particular highly beneficial with respect to the three agents identified, and are further considered illustrative of modalities for similar types of compounds. These embodiments are applied according to various aspects of the invention in novel administrations to provide effective results in preventing RCN when delivered locally to the renal arteries, or systemically in concert with selected local delivery.

Local Renal Delivery of Saline

Normal Saline (0.9% Sodium Chloride USP) for intravenous administration is typically used to provide a source of water and electrolytes, for extra-cellular fluid replacement, for treatment of metabolic alkalosis in the presence of fluid loss and mild sodium depletion, as a priming solution in hemodialysis procedures, to initiate and terminate blood transfusions without lysing red blood cells, and as a diluent for the infusion of compatible drug additives.

Currently, hydration is limited by the ability of the patient to process this extra fluid and insufficient time during the hospitalization to administer adequate volumes of fluid at a rate that is tolerable to the patient. Increases in central venous pressure due to fluid overload may not be tolerated, and fluid buildup in the lungs may occur, lowering hemoglobin oxygen saturation and thus exacerbating the hypoxic condition in the medullary kidney and elsewhere in the body.

However, with bi-lateral local delivery of saline to the renal arteries it can be seen that these issues can be mitigated by: (a) reducing the overall volume of saline needed to achieve the desired affect; (b) adding a mechanical “flushing” effect locally in the kidneys, as the volume needed to dilute the kidneys' share of the blood is a given percent is much less than that needed to dilute the patient's entire volume by the same percent; and (c) immediate removal of the infusate directly into the kidneys by the kidneys themselves, thus limiting the system's exposure to the infused fluid and reducing the potential for systemic fluid overload. The kidneys excrete saline very efficiently. Therefore, relatively high volumes of saline could potentially be injected during the catheterization procedure intra-renally, such that circulating volume is not significantly altered while the therapeutic and practical utility of saline can be maximized.

A low profile, self-cannulating bifurcated renal catheter provides one beneficial mechanism to allow for an easy means to deliver the saline locally to the renal arteries, with placement possible under fluoroscopy without the need for other guide wires or catheters. In one particular embodiment, the catheter delivers the saline locally before, during, and after the introduction of radiocontrast for arterial visualization or other procedures.

One highly beneficial mode of treatment for saline includes the pre-procedural systemic hydration up to a maximum level tolerated by the patient without signs of dangerously elevated CVP, pulmonary edema, or reduced arterial oxygenation. Often this may include delivery of up to about 3 cc/kg/hr for up to about 24 hours pre-procedurally, but practical limitations of the patient's time in the hospital and ability to tolerate over-hydration may limit the time and dose given. Typically, the dose given may be determined by ramping up (titration) the infusion of saline until an undesirable systemic effect is seen, and then reducing the dose back to the highest level that demonstrated no adverse consequences.

During the catheterization, where fluoroscopy is necessarily available, in many instances it is normally desirable to give the saline directly, and up to a level that the kidneys can tolerate in terms of immediate removal. The dose level may be set empirically, or beneficially customized such that it is determined by titration. The catheter or infusion pump design may provide practical limitations to the amount of fluid that can be delivered; however, it would be desired to give as much as possible, and is contemplated that levels up to 2 liters per hour (about 25 cc/kg/hr in an average about 180 lb patient) may be beneficial. Again, after the procedure, systemic delivery could begin again, at the level stated above, for example up to about 24 hours within practical limits. Longer times may be used should the patient remain in the hospital, or shorter times to suit a particular need requiring less infusion. Although normal saline is preferred, half normal saline or other salines may be used depending on the needs and risk factors of the patient.

It will be seen that the only substantial foreseeable risk of intra-renal normal saline infusion is a remaining potential for systemic volume overload. In one beneficial embodiment, the total volume of intra-renal saline administered to the renal arteries is maintained at a level that is less than approximately 800 mL. This volume would likely be tolerated even if it were infused intravenously in most patients. To monitor fluid status during the procedure, systemic oxygenation (i.e. pulse oximetry) is preferably monitored as well as monitoring filling pressures (i.e. central venous pressure) and blood pressure. If circulatory overload occurs during the procedure, it can be treated with diuretics, supplemental oxygenation, or means to reduce pulmonary pressures such as morphine. Cardiac function can also be improved by the use of vasodilators such as nitroglycerin and/or inotropes, if needed. Further, equipment for intubation would be readily available should significant hypoxemia and respiratory distresses develop.

In any event, it can be seen that the foregoing embodiments for local saline delivery provide substantial benefit over prior methods that have been previously been considered “gold standards” for renal protection against RCN, in particular by incorporating the benefits of local bilateral renal delivery to the benefit of both the renal system, and the rest of the patient's system. Moreover, combined therapies with local and systemic windows, provide the benefit of customizing the solution to meet the different physiologic and patient management needs during different treatment periods.

Local Renal Delivery of Papaverine

Because of its ability to provide generic smooth muscle tonus relaxation, papaverine is a highly beneficial agent to increase blood flow in the medullary kidney and thus reduce the probability of radiocontrast-induced nephropathy from a contrast insult.

There is substantial experience in the interventional environment for the acute treatment of artificially induced vasospasm (contraction) using Papaverine. The present embodiments provide substantial improved benefits via the application of local papaverine delivery into the renal system, bi-laterally, thus utilizing the special vasodilatory properties as a highly localized means to reduce RCN bilaterally to the renal system.

Significant dose limitations exist with papaverine if given systemically over prolonged time exposures (i.e., the time of a percutaneous coronary intervention or “PCI”). Therefore, the local delivery modalities, via novel bi-lateral renal drug infusion systems and methods as described herein, provide a substantial leap forward, allowing papaverine to be used in this prophylactic or early therapeutic indication.

As discussed for saline, the clinical logistics of providing for local agent delivery to the renal arteries remote from the PCI or other catheterization environments make it difficult to specify this method of treatment; however, pre-, during, and/or post-procedural local papaverine delivery, or combinations thereof such as illustrated above by reference to FIG. 2, indeed provide highly beneficial dose delivery regimens in terms of clinical efficacy, that being a reduction in the incidence of RCN. That being said, additional regimens may be used to best maximize the clinical benefit of papaverine without causing undue hurdles in the clinical setting.

It is thus contemplated according to further embodiments that a very low, systemic dose of papaverine may be given, either alone or in conjunction with other medical management such as for example saline loading, prior to the anticipated contrast insult. Such a dose may be on the order, for example, of between about 3 to about 14 mg/hr (based on bolus indications of approximately 1040 mg about every 3 hours—papaverine is not generally dosed by weight). In an alternative embodiment, a dosing of 2-3 mg/min or 120-180 mg/hr is provided.

Notwithstanding the particular benefit of this dosing range for this period, it is also believed that higher doses delivered locally would be safe. Titration is a further mechanism believed to provide the ability to test a patient for tolerance to higher doses.

During the time of the catheterization, local delivery would be favored and easily achievable with the bifurcated catheter design discussed earlier. Local dosing of up to about 4 mg/min (again, not typically weight-based) has been safely demonstrated in certain in-vivo animal studies, and local renal doses of about 2 mg/min and about 3 mg/min have been shown to increase renal blood flow rates in human subjects. It is thus appropriately understood that local bilateral renal delivery of papaverine will help to reduce the risk of RCN in patients with pre-existing risk factors such as high baseline serum creatinine, diabetes mellitus, or other demonstration of compromised kidney function. It is believed that these doses are safe, and titration may be employed to explore higher doses in patients whose medical condition warrants such additional protection.

Post-procedure, again the local administration route is considered a highly beneficial mode. Dosage levels may be for example consistent with peri-procedural ranges should this continued local administration be chosen for a particular patient. However, systemic administration may be the modality of choice for a particular therapy, such as for example at the pre-procedural dosing levels, and possibly in conjunction with hydration as before.

Local Renal Delivery of Fenoldopam

Fenoldopam mesylate is a commercially available short-acting dopamine-1 (DA-1) specific agonist. The approved use for fenoldopam is for the in-hospital intravenous treatment of hypertension when rapid, but quickly reversible, blood pressure lowering is needed. Fenoldopam causes dose-dependent renal vasodilation at systemic doses as low as approximately 0.01 mcg/kg/min through approximately 0.5 mcg/kg/min IV and it increases blood flow both to the renal cortex and to the renal medulla. Due to this physiology, fenoldopam may be utilized for protection of the kidneys from ischemic insults such as high-risk surgical procedures and contrast nephropathy. Fenoldopam is considered a beneficial agent for this application as it a vasodilator (specifically, a dopamine D₁-like receptor agonist) with recognized effects on the capillaries of the kidney's medullary region. For this reason, it is quite promising for study in prevention of RCN. However, problems may arise from purely systemic delivery. Fenoldopam, like papaverine, is a vasodilator and therefore also has the potential for creating a dangerous systemic hypotensive condition in doses that might be necessary to adequately affect the renal medullary vasculature.

Accordingly, the use of fenoldopam by systemic infusion is limited by dose-dependent hypotension, which is mediated by DA-1 induced systemic vasodilation. Administration of fenoldopam to hypovolemic dogs receiving contrast prevented reduction in both renal blood flow and renal function (as measured by glomerular filtration rate). In a number of small studies, including a randomized, double blind, placebo-controlled trial, fenoldopam attenuated worsening of renal function in high-risk patients undergoing contrast procedures. These studies used fenoldopam doses between 0.05-0.5 mcg/kg/min, typically administered for a total of 4-6 hours and started prior to contrast administration. One randomized, double blind, placebo-controlled trial (CONTRAST) of 300 patients found no benefit of 0.05-0.1 mcg/kg/min of systemic IV fenoldopam over saline given over 12 hours.

Recent data, however, suggest that 0.2 mcg/kg/min of fenoldopam has greater efficacy than 0.1 mcg/kg/min in preventing contrast nephropathy, albeit with a higher risk of hypotension. It should be noted that the renal dose-response curve for fenoldopam was derived from a population of normal volunteers and not in a population of patients with renal dysfunction, for whom higher doses may be required to elicit the blood flow effects seen in normal patients. Indeed, whereas in normal volunteers, a dose of 0.1 mcg/kg/min increased renal blood flow by approximately 40% from baseline, in patients with a mean serum creatinine of 2.6 mg/dL, the increment in renal blood flow at that dose was only 16%. It is plausible, therefore, that patients in the CONTRAST trial, who were selected to have renal insufficiency, were not given sufficient doses of fenoldopam for efficacy.

It can be seen therefore that intra-renal administration of fenoldopam may allow lower systemic but higher local fenoldopam dosing, thereby taking advantage of its dose-dependent effects on renal blood flow. Ninety percent of infused fenoldopam is eliminated in the urine. Regional delivery of fenoldopam at effective concentrations at the kidney reduces the occurrence of side effects experienced with systemic administrations. The most common side effects are headache, nausea and vomiting, cutaneous flushing, hypotension, (reflex) tachycardia, hypokalemia, non-specific ST segment and T-wave changes, dizziness, and dyspnea. All of the above have been observed to occur, in systemic regimens, with a frequency of 5% and lower with the exception of headache, which has been observed to occur with a frequency of 11%. Fenoldopam, like dopamine, increases intra-ocular pressure, although the clinical significance of this is unknown. Fenoldopam also contains the preservative sodium metabisulfite that may elicit allergic reactions from some patients with sulfite allergies.

The routine monitoring performed in the cardiac catheterization laboratory will be sufficient to monitor for fenoldopam side effects such as hypotension and tachycardia, both of which occur in a dose-dependent manner. Statistically significant tachycardia was not seen in clinical trials at intravenous doses of less than 0.3 mcg/kg/min, which is a higher dose than will often be required to reach clinical efficacy for bilateral local renal delivery. Lowering blood pressure to an undesirable extent can generally be reversed by cessation of the fenoldopam infusion and other conservative measures such as changes in body position. Additional treatments such as intravenous fluid and vasopressor drugs could be easily administered in the cardiac catheterization laboratory, if needed. Likewise, fenoldopam-induced reflex tachycardia of a clinically important degree could be managed with blood pressure management as above, or potentially, with use of β-receptor antagonists, which are also readily available in the catheterization laboratory. Blood tests to evaluate serum potassium may be undertaken, and patients with a history of glaucoma or sulfite allergy might be excluded from indicated treatment with Fenoldopam in certain circumstances. However, again the benefits of truly local delivery and reduced systemic effects may render even these patients treatable with fenoldopam according to the systems and methods herein described.

Thus, local delivery is provided according to the embodiments herein, with specified dosing regimes described in further embodiments for managing the patient pre-, during, and post-procedurally. Again, highly beneficial modes of agent administration distributed locally—before, during, and after the contrast insult—can maximize the potential protective benefit of fenoldopam.

However, access to fluoroscopy would again be a limiting logistical factor, and thus the pre-procedural administration may in many circumstances be necessarily systemic. Up to about four hours of pre-procedural administration is suggested according to one further more detailed mode, and at least about 15-30 minutes may be a typical pre-procedural window to ensure a steady-state level is achieved. This systemic administration may be combined with over-hydration to maximize any effect. Dosing from approximately 0.01 to approximately 3.2 mcg/kg/min, or 0.05 to 1.6 mcg/kg/min to each kidney is considered suitable for most patients. As before, it is likely beneficial in many instances to pick a starting dose and titrate up or down as required to determine a patient's maximum tolerated systemic dose.

At the time of the procedure, a switch to local delivery can easily be made with the previously described bifurcated catheter. At this point the dose may most likely be raised substantially, due to a drop-off in systemic effects. Animal safety testing has been conducted to demonstrate safety at dosages of about 0.2 mcg/kg/min for about four-hour infusions. In analogous fashion to the human data discussed above with papaverine, intra-renal delivery of fenoldopam at about 0.1 mcg/kg/min and about 0.2 mcg/kg/min is sufficient to provide significant and immediate increases in renal artery blood flow, indicating that positive effects may be occurring downstream (i.e., in the medullary capillary beds). Therefore another titration (in this situation most frequently migrating upward) is performed according to further embodiments, starting at the systemic dose, and moving towards a tolerated local dose, for the duration of the intended catheterization. Post-procedure, if the specialized bifurcated catheter is preferably to be removed, then the administration could be returned to a systemic dose, with or without additional hydration concurrently. The post-procedural administration may be beneficial for up to about 24 hours for example, though about 12 hours may be sufficient, as residual contrast-induced vasospasm will generally have cleared by that time.

Other dose delivery modalities may be applicable, other than those specifically mentioned for the various fluid agents above. For example, it may be beneficial in any or all cases, pre-, during, or post-procedural, whether mentioned above or not, to combine the given systemic or local agent administration with additional fluid. This applies even in the case of saline, which, for example, may be given both locally and systemically simultaneously, which may be considered clinically beneficial in many cases, such as in terms of certain RCN treatments or some other condition. Similarly, there may be benefit in terms of efficacy in RCN reduction or other clinical condition to delivering a combined local and systemic administration of the same agent or of multiple agents (i.e., local and systemic fenoldopam concurrently, or local papaverine combined with systemic fenoldopam).

It is to be appreciated that the compound agents specified herein are considered highly beneficial. However, other agents may be chosen and used in similar modes, in particular where their bioactivity and safety are demonstrated at these delivery modes equivalent to the agents specified herein. For example, precursors such as pro-drugs that are metabolized to form the active agent may be used. Or, other derivatives or analogs, such as modifying the molecules in manners not substantially affecting their intended activity according to the specified embodiments, may be made. In addition, it is to be appreciated that the fluid agents specified herein may be illustrative of classes of agents having similar properties, e.g. such as vasodilators for example, of which other specific agents of similar type may be used in the settings disclosed hereunder. Even where activities may vary from the specified agents here, the particular dose delivery regimens, such as amounts and time periods, may be modified according to one of ordinary skill without undue experimentation in order to accomplish the desired results without departing from the intended scope hereof.

For example, simple pre-clinical animal experimentation observing differing particular variables for optimal ranges, followed by standard protocols to gain clinical experience with the optimized parameters learned from the pre-clinical experiments (and often followed by further refinements), would be considered a suitable mechanism by which to customize the broad aspects presented hereunder to a particular compound or indication. Such results are considered within the intended scope hereunder, which is intended to apply broadly to various applications notwithstanding further refinements that may be required in the normal course of applied development. In this regard, while the particular dose regimes are considered independently highly beneficial, such are not intended to be limiting in all cases, and modifications may be made for particular settings of use without departing from the intended broad scope of certain aspects of the invention.

Moreover, it is also described above that the various dose delivery modes for the identified agents and compounds be accomplished in certain regards according to a bifurcated, bi-lateral intra-renal drug delivery catheter. However other devices may be utilized to accomplish similar results in terms of being able to provide bilateral, local, renal drug delivery simultaneously into both kidneys in a manner beneficial when compared to systemic dosing.

Although the description above contains many details, these should not be construed as limiting the scope of the invention but as merely providing illustrations of some of the presently preferred embodiments of this invention. Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

1. A method for preventing radiocontrast induced nephropathy in a patient in response to delivering radiocontrast agent to the patient, comprising:
locally delivering a therapeutic dose of a renal therapy agent bi-laterally to the renal arteries of a patient during exposure to the radiocontrast agent; and
monitoring the renal function of the patient.

2. A method as recited in claim 1, wherein said agent comprises a vasodilator.

3. A method as recited in claim 2, wherein said vasodilator comprises fenoldopam mesylate or an analogue or derivative thereof.

4. A method as recited in claim 3, wherein said therapeutic dose comprises an administration rate of between approximately 0.01 mcg/kg/min to approximately 3.2 mcg/kg/min.

5. A method as recited in claim 3, wherein said therapeutic dose comprises an administration rate of between approximately 0.1 mcg/kg/min to approximately 0.2 mcg/kg/min.

6. A method as recited in claim 2, wherein said vasodilator comprises a natriuretic He or an analogue or derivative thereof.

7. A method as recited in claim 6, wherein said dose of papaverine is administered at a rate of between about 2 mg/min to about 3 mg/min.

8. A method as recited in claim 1, wherein said agent comprises a hydrating agent, wherein said hydrating agent causes hydration of a patient over a normal baseline.

9. A method as recited in claim 8, wherein said hydrating agent comprises normal saline.

10. A method as recited in claim 9, wherein said therapeutic dose of normal saline comprises an administration rate of between about 20 cc/kg/hour to about 30 cc/kg/hour.

11. A method as recited in claim 9, wherein said therapeutic dose of normal saline comprises an administration rate of between about 24 cc/kg/hour to about 26 cc/kg/hour.

12. A method as recited in claim 1, wherein said renal function is monitored by periodically evaluating serum creatinine levels over time.

13. A method for preventing radiocontrast induced nephropathy in a patient, comprising:
delivering a first therapeutic dose of a first renal therapy agent to the patient during a first period that is before exposure to a radiocontrast agent; and
locally delivering a second therapeutic dose of a second renal therapy agent bi-laterally to the renal arteries of the patient during a second period that is during exposure to the radiocontrast agent.

14-131. (canceled)

132. A bilateral local renal therapy system for protecting a renal system from radiocontrast nephropathy associated with delivery of a radiocontrast agent within a vascular system of a patient, comprising:
a catheter having a proximal end and a distal end;
a fluid agent source comprising a fluid agent, the fluid agent source coupled with the proximal end of the catheter via a fluid agent delivery port; and
first and second tips coupled with the distal end of the catheter, the first and second tips in fluid communication with the fluid agent delivery port, and configured for bilateral entry into renal arteries of the patient.

133. The renal therapy system according to claim 132, wherein the fluid agent comprises saline.

134. The renal therapy system according to claim 132, wherein the fluid agent comprises a vasodilator.

135. The renal therapy system according to claim 134, wherein the vasodilator comprises a natriuretic or an analogue, derivative, or precursor thereof.

136. The renal therapy system according to claim 134, wherein the vasodilator comprises fenoldopam or an analogue, derivative, or precursor thereof.

137. The renal therapy system according to claim 132, further comprising:
a second fluid agent source comprising a second fluid agent, the second fluid agent source coupled with the proximal end of the catheter via a second fluid agent delivery port;
wherein the first and second tips are in fluid communication with the second fluid agent delivery port.

138. The renal therapy system according to claim 132, further comprising an infusion pump.
